(19) United States
(12) Patent Application Publication
(10) Pub. No.: US 2022/0064135 A1
(43) Pub. Date: Mar. 3, 2022

(54) Method for Preparation of 1,4-Sorbitan in Aqueous Medium

(71) Applicants: LONZA GUANGZHOU PHARMACEUTICAL LTD., Guangzhou, Guangdong (CN); Lonza Ltd, Visp (CH)

(72) Inventors: Jieping Wei, Guangdong (CN); Gesa Paradies, Brig (CH); Benjamin Wyler, Bern (CH); Dieter Scherer, Laufen (CH); Yanling Yang, Guangdong (CN); Xiaolong Zhang, Guangdong (CN); Weicheng Jiang, Guangdong (CN); Reta Zhu, Guangdong (CN)

(21) Appl. No.: 17/423,193

(22) PCT Filed: Jan. 16, 2020
(86) PCT No.: PCT/EP2020/051059

§ 371 Date: Jul. 15, 2021

Related U.S. Application Data
(60) Provisional application No. 62/799,821, filed on Feb. 1, 2019.

(30) Foreign Application Priority Data
Jan. 16, 2019 (CN) PCT/CN2019/071900
Feb. 1, 2019 (EP) 19154950.0
Feb. 13, 2019 (EP) 19157025.8
Feb. 13, 2019 (EP) 19157027.4

(51) Int. Cl. C07D 307/20

(52) U.S. Cl. CPC C07D 307/20

(57) ABSTRACT

The invention discloses a method for preparation of 1,4-sorbitan by dehydration of D-sorbitol in aqueous medium, wherein one equivalent of water is removed and a cyclization occurs, followed by a treatment with ethanol and isopropanol.

The invention discloses a method for preparation of 1,4-sorbitan by dehydration of D-sorbitol in aqueous medium, wherein one equivalent of water is removed and a cyclization occurs, followed by a treatment with ethanol and isopropanol.

### BACKGROUND OF THE INVENTION

1,4-Sorbitan is used for the production of pharmaceuticals, such as certain prostaglandin analogues, and for the production of excipients used in formulation of pharmaceuticals, such as Polysorbate 80.

S. Stolzberg, J. Am. Chem. Soc., 1946, 68, 919-921, discloses a method for preparation of 1,4-sorbitan by a dehydration of 100 g sorbitol in the presence of concentrated sulfuric acid and water at ca. 140° C. for 30 min, the method has a recrystallization step from isopropanol as a last step, reported yield is 33 g; the calculated molar yield is 36.6%.

CN 101948451 A discloses a method for preparation of high-purity 1,4-sorbitan, which is characterized by taking sorbitol as a raw material through two times of dehydration and three times of crystallization. Already after the second dehydration, a base is added to the reaction mixture for neutralization, then the reaction mixture is filtered to remove an acid catalyst used in the second dehydration reaction, the a decolourization is done by addition of activated carbon, which again necessitates a filtration for removing the activated carbon. The crystallization is done with methanol, after each crystallization step a filtration is done. The content of 1,4-sorbitan is 73.7% after the decolourization, 87% after the first, 94% after the second and 99.2% after the third crystallization. The yield after the decolourization was 70%, the yield after the three crystallization was 19%, so the overall yield was 13%.

Any use of a compound for or in pharmaceutical applications requires a defined purity and usually also a high purity.

There was a need for a method for preparation 1,4-sorbitan with high yield, high purity, low content of isosorbide or D-sorbitol; the method should be as economic as possible, such as with a low number of steps such as filtration or with a low number of different chemicals used, also the method should be suited to be done “in one pot”, meaning that only one reactor can be used.

Unexpectedly, a method was found which gives high yield, high purity, low content of isosorbide, low content D-sorbitol; the method is economic, has a low number of steps such as filtration and uses a low number of different chemicals. The method can be done in one reactor.

### ABBREVIATIONS

equiv, eq equivalent

Isosorbide compound of formula (3), MW 146.1 g/mol, CAS 652-67-5

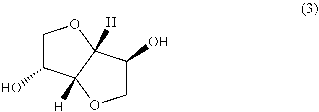

1,4-Sorbitan compound of formula (1), MW 164.2 g/mol, CAS 27299-12-3

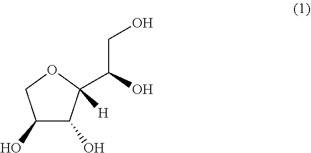 [SMILES: OC[C@@H](O)[C@H]1OC[C@H](O)[C@H]1O]

D-Sorbitol compound of formula (2), MW 182.2 g/mol, CAS 50-70-4

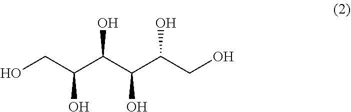 [SMILES: OC[C@@H](O)[C@@H](O)[C@H](O)[C@@H](O)CO]

MW molecular weight

TBAB Tetrabutylammonium bromide

% percent are percent by weight (wt %), if not stated otherwise

### SUMMARY OF THE INVENTION

Subject of the invention is a method for preparation of 1,4-sorbitan with three consecutive steps STEP1, STEP2 and STEP3, wherein in STEP1 D-sorbitol is dehydrated in a dehydration reaction DEHYDREAC in the presence of p-toluenesulfonic acid and tetrabutylammonium bromide, STEP1 provides a mixture MIX1;

in STEP2 ethanol is mixed with MIX1, STEP2 provides a mixture MIX2;

in STEP3 isopropanol is mixed with MIX2, STEP3 provides a mixture MIX3;

D-sorbitol is used for STEP1 in form of a mixture of D-sorbitol with water.

### DETAILED DESCRIPTION OF THE INVENTION

Preferably, D-sorbitol is used for and charged in STEP1 in form of a mixture of D-sorbitol with water.

The mixture of D-sorbitol with water which is used for STEP1 can be a solution or a suspension of D-sorbitol in water.

Preferably, D-sorbitol is used for STEP1 as a mixture of D-sorbitol with water with a content of D-sorbitol of from 20 to 80 wt %, more preferably of from 40 to 80 wt %, even more preferably of from 60 to 80 wt %, especially of from 65 to 75 wt %, in particular of 70 wt %, of D-sorbitol, the wt % being based on the total weight of the mixture of D-sorbitol with water.

Preferably, TBAB is used for STEP1 as a mixture of TBAB with water; more preferably, TBAB is used for and charged in STEP1 as a mixture of TBAB with water. The mixture of TBAB with water can be a solution or a suspension of TBAB in water.

More preferably, TBAB is used for STEP las a mixture of TBAB with water with a content of TBAB of from 20 to 80 wt %, even more preferably of from 40 to 80 wt %, especially of from 60 to 80 wt %, more especially of from 60 to 75 wt %, even more especially of from 60 to 70 wt %, in particular of 65 wt %, of TBAB, the wt % being based on the total weight of the mixture of TBAB with water.

Preferably, STEP1 comprises three steps STEP1A, STEP1B and STEP1C.

In STEP1A a mixture of D-sorbitol with water, TBAB and p-toluenesulfonic acid are mixed providing a mixture MIXT1A;

in STEP1B water is distilled off in a distillation DIST1A from MIX1A, providing a mixture MIX1B;

in STEP1C MIX1B is stirred providing MIX1.

MIX1A comprises D-sorbitol, TBAB and water.

Preferably, DIST1A is done at a temperature TEMP1A of from 40 to 100° C., more preferably of from 50 to 90° C., even more preferably of from 55 to 85° C., in particular of from 60 to 80° C.

Preferably, DIST1A is done at reduced pressure PRESS1A; PRESS1A is adjusted in such a way that DIST1A takes place at TEMP1A.

Preferably, all water is distilled off from MIX1A in STEP1A.

Preferably, DIST1A is done for such a time period until all water is distilled off from MIX1A.

Preferably, in STEP1C the stirring of MIX1B is done at a temperature TEMP1C; TEMP1C is from 80 to 120° C.

Preferably, TEMP1C is from 90 to 110° C., more preferably from 100 to 110° C., in particular 105° C.

Preferably, in STEP1C the stirring of MIX1B is done for a time TIME1C providing MIX1, TIME1C is from 2 to 10 h.

Preferably, TIME1C is from 4 to 8 h, more preferably from 5 to 7 h, in particular 6 h.

Preferably, the stirring during TIME1C is done under reduced pressure PRESS1C; in one embodiment PRESS1C is adjusted so the stirring is done stirred under reflux conditions at the chosen TEMP1C, in another embodiment, PRESS1C is from 40 to 100 mbar, more preferably from 40 to 60 mbar, in particular 50 mbar.

Preferably, after TIME1C the pressure is brought back from PRESS1C to atmospheric pressure by insertion of nitrogen.

Preferably, STEP2, STEP3 and STEP4 are done at atmospheric pressure.

Preferably, the p-toluene sulfonic acid is used in form of p-toluenesulfonic acid monohydrate; so in any embodiment where p-toluene sulfonic acid is mentioned, the preferred embodiment is p-toluenesulfonic acid monohydrate.

DEHYDREAC takes place in STEP1B, in STEP1C or in both;

preferably DEHYDREAC takes place in STEP1B and can also extend into STEP1C.

Preferably, no organic solvent, more preferably no solvent except water, is present in or used for DEHYDREAC.

Preferably, no organic solvent, more preferably no solvent except water, is present in or used for STEP1.

Preferably, in DEHYDREAC only the three components D-sorbitol, p-toluenesulfonic acid and tetrabutylammonium bromide are used for and are charged for DEHYDREAC, with the D-sorbitol being used and charged in form of a mixture of D-sorbitol with water, more preferably also with the TBAB being used and charged in form of a mixture of TBAB with water.

Preferably, the molar equivalent of p-toluenesulfonic acid in DEHYDREAC acid is from 0.2 to 1.6%, more preferably from 0.4 to 1.4%, even more preferably from 0.6 to 1.2%, especially from 0.6 to 1.0%, more especially from 0.8 to 1.0%, in particular 0.9%, of the molar equivalents of D-sorbitol.

Preferably, the molar equivalent of tetrabutylammonium bromide in DEHYDREAC acid is from 1 to 3%, more preferably from 1.2 to 2.5%, even more preferably from 1.4 to 2%, especially from 1.6 to 1.8%, in particular 1.7%, of the molar equivalents of D-sorbitol.

Preferably, the weight of ethanol mixed in STEP2 is from 0.2 to 5 fold, more preferably from 0.2 to 2 fold, even more preferably from 0.2 to 1 fold, especially from 0.2 to 0.8 fold, more especially from 0.2 to 0.6 fold, even more especially from 0.3 to 0.5 fold, in particular 0.4 fold, of the weight of D-sorbitol.

Preferably, the weight of isopropanol mixed in STEP2 is from 0.2 to 5 fold, more preferably from 0.2 to 2 fold, even more preferably from 0.2 to 1 fold, especially from 0.2 to 0.8 fold, more especially from 0.2 to 0.6 fold, even more especially from 0.3 to 0.5 fold, in particular 0.4 fold, of the weight of D-sorbitol.

Preferably, STEP2 is done at a temperature TEMP2 of from 60 to 90° C., more preferably of from 60 to 85° C., even more preferably of from 65 to 80° C., in particular of from 70 to 75° C.

Preferably, STEP1 comprises a cooling COOL1 after DEHYDREAC, preferably after STEP1C, where MIX1 is cooled from TEMP1C to TEMP2.

Preferably, COOL1 is done in a time TIME1-2, TIME1-2 is from 10 min to 10 h, more preferably from 15 min to 5 h, even more preferably from 15 min to 2 h, especially from 20 min to 1.5 h, more especially from 30 to 60 min, in particular 45 min.

If STEP1 comprises COOL1 and SETP1C has been done at PRESS1C, then the pressure can be brought back from PRESS1C to atmospheric pressure before, during or after COOL1.

Preferably, after the mixing of ethanol with MIX1, STEP2 comprises a stirring STIRR2 of MIX2 for a time TIME2-1, TIME2-1 is from 30 min to 10 h, more preferably of from 1 to 8 h, even more preferably of from 1 to 6 h, especially from 1 to 4 h, more especially from 1.5 to 3 h, in particular 2 h.

Preferably, STIRR2 is done at TEMP2.

Preferably, crystal seed of 1,4-sorbitan is added to MIX2;

preferably, of from 0.1 to 2 wt %, more preferably of from 0.2 to 1.5 wt %, even more preferably of from 0.3 to 1 wt %, especially of from 0.4 to 0.7 wt %, in particular 0.5 wt %, of crystal seed of 1,4-sorbitan are added, the wt % being based on the weight of D-sorbitol;

preferably, crystal seed of 1,4-sorbitan is added to MIX2 after STIRR2.

Preferably, MIX2 is a clear solution;

more preferably, MIX2 is a clear solution before the addition of crystal seed of 1,4-sorbitan;

more preferably, MIX2 after STIRR2 is a clear solution;

even more preferably, MIX2 after STIRR2 and before an addition of crystal seed of 1,4-sorbitan to MIX2 is a clear solution.

Preferably, the mixing of isopropanol with MIX2 in STEP3 is done at a temperature TEMP3-1 of from 20 to 70° C., more preferably of from 30 to 60° C., even more preferably of from 40 to 55° C., in particular of from 45 to 50° C.

Preferably after the mixing of ethanol with MIX1, STEP2 comprises a cooling COOL2, where MIX2 is cooled from TEMP1C or TEMP2 to TEMP3-1.

Preferably, COOL2 is done after STIRR2.

More preferably, COOL2 is done after an addition of crystal seed of 1,4-sorbitan to MIX2.

Preferably, COOL2 is done from TEMP2 to TEMP3-1.

Preferably, STEP2 comprises STIRR2 and an addition of crystal seed of 1,4-sorbitan to MIX2 and COOL2, and COOL2 is done after an addition of crystal seed of 1,4-sorbitan to MIX2.

Preferably, COOL2 is done in a time TIME2-2, TIME2-2 is from 1 to 10 h, more preferably from 1 to 8 h, even more preferably from 1 to 6 h, especially from 1 to 4 h, more especially from 1 to 3 h, in particular 2 h.

Preferably, crystal seed of 1,4-sorbitan is added to MIX2 after STIRR2 and before COOL2.

Preferably, the amount of ethanol used in STEP2 is such that after the mixing of ethanol with MIX1 a clear solution of 1,4-sorbitan in ethanol, preferably at TEMP2, is obtained;

preferably the amount of ethanol is such that said clear solution is a clear solution of 1,4-sorbitan in ethanol at TEMP2 and an oversaturated solution at of 1,4-sorbitan in ethanol at temperatures under TEMP2, preferably such as TEMP3-2, more preferably such as TEMP3-1;

more preferably the amount of ethanol is such that said clear solution is an oversaturated solution of 1,4-sorbitan in ethanol at TEMP2.

Preferably said clear solution is obtained after STIRR2; more preferably after STIRR2 and before an addition of crystal seed of 1,4-sorbitan to MIX2.

Preferably, the amount of ethanol is such that crystallization starts during COOL2; more preferably, the amount of ethanol is such that
after the mixing of ethanol with MIX1 a clear solution of 1,4-sorbitan in ethanol, preferably at TEMP2, is obtained; and
the crystallization starts during COOL2;

even more preferably, the amount of ethanol is such that
after the mixing of ethanol with MIX1 a clear solution of 1,4-sorbitan in ethanol, preferably at TEMP2, is obtained; and
that said clear solution is a clear solution of 1,4-sorbitan in ethanol at TEMP2 and an oversaturated solution at of 1,4-sorbitan in ethanol at temperatures under TEMP2, preferably such as TEMP3-2, more preferably such as TEMP3-1; and
that crystallization starts during COOL2.

Preferably, MIX2 after COOL2 is a suspension.

Preferably, after the mixing of isopropanol with MIX2, STEP3 comprises a cooling COOL3 of MIX3 to a temperature TEMP3-2 of from −5 to 10° C., more preferably of from −2.5 to 7.5° C., even more preferably of from −1 to 6° C., in particular of from 0 to 5° C.

Preferably, COOL3 is done in a time TIME3-1, TIME3-1 is from 1 to 10 h, more preferably of from 1 to 8 h, even more preferably of from 1 to 6 h, especially from 2 to 6 h, more especially from 2 to 4 h, in particular 3 h.

Preferably, after the mixing of isopropanol with MIX2, STEP3 comprises a stirring STIRR3 of MIX3.

Preferably, STIRR3 is done at TEMP3-2.

Preferably, STIRR3 is done for a time TIME3-2, TIME3-2 is from 1 to 12 h, more preferably from 1 to 10 h, even more preferably from 1 to 8 h, especially from 2 to 6 h, more especially from 3 to 5 h, in particular 4 h.

Preferably, STIRR3 is done after COOL3.

More preferably, STIRR3 is done after COOL3 and STIRR3 is done at TEMP3-2.

Preferably, MIX3 is a suspension.

Preferably, the method comprises a STEP4, STEP4 is done after STEP3, in STEP4 1,4-sorbitan is isolated from MIX3.

The isolation in STEP4 of 1,4-sorbitan from MIX3 can be done by any means known to the skilled person, such as evaporation of any liquids in MIX3, filtration, centrifugation, drying, or a combination thereof, preferably the isolation is done by filtration.

Preferably, 1,4-sorbitan is isolated in STEP4 from MIX3 by filtration providing a presscake, preferably followed by washing the presscake with isopropanol, preferably followed by drying of the washed presscake, preferably the drying takes place at a temperature of from 30 to 70° C., more preferably of from 35 to 65° C., even more preferably of from 40 to 60° C., in particular of from 45 to 55° C.

In one embodiment,

STEP1 comprises consecutively DEHYDREAC and COOL1;

STEP2 comprises after the mixing of ethanol consecutively STIRR2 and COOL2;

STEP3 comprises after the mixing of isopropanol consecutively COOL3 and STIRR3;

preferably,

STEP1 comprises consecutively STEP1A, STEP1B, STEP1C and COOL1;

STEP2 comprises after the mixing of ethanol consecutively STIRR2 and COOL2;

STEP3 comprises after the mixing of isopropanol consecutively COOL3 and STIRR3.

more preferably,

STEP1 comprises consecutively STEP1A, STEP1B, STEP1C and COOL1;

STEP2 comprises after the mixing of ethanol consecutively STIRR2, the addition of crystal seed of 1,4-sorbitan to MIX2, and COOL2;

STEP3 comprises after the mixing of isopropanol consecutively COOL3 and STIRR3.

Preferably, STEP1, STEP2 and STEP3 are done consecutively in one and the same reactor.

### EXAMPLE

Materials

The materials were used in the following qualities, if not otherwise stated:

| | |
|---|---|
| TsOH—H₂O | 99 wt % |
| Ethanol | 99 wt % |
| Isopropanol | 99 wt % |

GC Method

Instrument Parameters

Column DB-1 HT (30 m*0.25 mm*0.1 μm) Agilent Technologies, Santa Clara, USA

Temperature Program:

| | |
|---|---|
| Initial; time | 100° C.; 0 min |
| Rate1; Final 1; Time 1 | 8° C./min; 350° C.; keep 10 min |
| Run Time | 41.25 min |
| Equilibration Time | 0.5 min |
| Mode | Cons. flow |
| Carrier gas | H₂ |
| Flow | 1.5 ml/min |
| Split ratio | 10:1 |
| Inlet Temperature | 350° C. |
| Injection Volumn | 1 microliter |
| Detector temperature | 350° C. |

Sample Preparation

Sample Stock Solution

Add 2 g sample to 5 ml pyridine and 10 ml acetic anhydride in a screw-cap bottle (25 mL) and heat up to 120° C. for 2 hours under stirring.

Sample Solution

0.5 ml of Sample stock solution is added into an autosampler vial with 1 ml of dichloromethane and mixed

1,4-Sorbitan is detected at ca. 12.3 min.

### Example 1

500 g of an aqueous solution with 70 wt % of D-sorbitol were charged into a reactor A, then 3.17 g p-Toluenesulfonic acid monohydrate were charged, then 16.52 g of an aqueous solution with 65 wt % TBAB were charged. Then water was distilled off at 60 to 80° C. under reduced pressure by gradually increasing the vacuum until all the water was distilled off. The reaction mixture was stirred at 300 rpm at 105° C. under reduced pressure of 50 mbar for 6 h. Then the vacuum was broken by insertion of N₂ to 1 bar.

The mixture was cooled to 70 to 75° C. in ca. 45 min. 141.61 g of EtOH were charged. The mixture was stirred at 70 to 75° C. for 2 h. A clear solution was obtained. 1.58 g of crystal seed of 1,4-sorbitan were charged. The mixture was cooled to 45 to 50° C. within 2 h. During this time of cooling to 45 to 50° C. crystallization set in. 141.44 g of i-PrOH were charged. The mixture was cooled to 0 to 5° C. within 3 h. The mixture was stirred at 0 to 5° C. for 4 h. The mixture was filtered. The presscake was washed with 141.44 g of i-PrOH. The presscake was dried at 45 to 55° C. under vacuum for 20 h.

141.95 g of 1,4-sorbitan were obtained.

The yield was 45%.

GC Area-%:

| | |
|---|---|
| 1,4-Sorbitan | 97.9% |
| Isosorbide | 0.09% |
| D-Sorbitol | 0.10% |

1. A method for preparation of 1,4-sorbitan with three consecutive steps STEP1, STEP2 and STEP3, wherein
in STEP1 D-sorbitol is dehydrated in a dehydration reaction DEHYDREAC in the presence of p-toluenesulfonic acid and tetrabutylammonium bromide, STEP1 provides a mixture MIX1;
in STEP2 ethanol is mixed with MIX1, STEP2 provides a mixture MIX2;
in STEP3 isopropanol is mixed with MIX2, STEP3 provides a mixture MIX3;
D-sorbitol is used for STEP1 in form of a mixture of D-sorbitol with water.

2. The method according to claim 1, wherein
TBAB is used for STEP1 as a mixture of TBAB with water.

3. The method according to claim 1, wherein
STEP1 comprises three steps STEP1A, STEP1B and STEP1C;
in STEP1A a mixture of D-sorbitol with water, TBAB and p-toluenesulfonic acid are charged providing a mixture MIXT1A;
in STEP1B water is distilled off in a distillation DIST1A from MIX1A, providing a mixture MIX1B;
in STEP1C MIX1B is stirred providing MIX1.

4. The method according to claim 3, wherein
all water is distilled off from MIX1A in STEP1A.

5. The method according to claim 3, wherein
in STEP1C the stirring of MIX1B is done at a temperature TEMP1C;
TEMP1C is from 80 to 120° C.

6. The method according to claim 1, wherein no organic solvent is present in or used for DEHYDREAC.

7. The method according to claim 1, wherein the molar equivalent of p-toluenesulfonic acid in DEHYDREAC acid is from 0.2 to 1.6%, of the molar equivalents of D-sorbitol.

8. The method according to claim 1, wherein the molar equivalent of tetrabutylammonium bromide in DEHYDREAC acid is from 1 to 3%, of the molar equivalents of D-sorbitol.

9. The method according to claim 1, wherein the weight of ethanol mixed in STEP2 is from 0.2 to 5 fold, of the weight of D-sorbitol.

10. The method according to claim 1, wherein the weight of isopropanol mixed in STEP2 is from 0.2 to 5 fold, of the weight of D-sorbitol.

11. The method according to claim 1, wherein STEP2 is done at a temperature TEMP2 of from 60 to 90° C.

12. The method according to claim 1, wherein MIX2 is a clear solution.

13. The method according to claim 1, wherein crystal seed of 1,4-sorbitan is added to MIX2.

14. The method according to claim 1, wherein the mixing of isopropanol with MIX2 in STEP3 is done at a temperature TEMP3-1 of from 20 to 70° C.

15. The method according to claim 1, wherein after the mixing of isopropanol with MIX2, STEP3 comprises a stirring STIRR3 of MIX3 at a temperature TEMP3-2 of from −5 to 10° C.

16. A method for preparing polysorbate comprising preparing 1,4-sorbitan according to claim 1.

17. A polysorbate prepared by the process according to claim 1.
